(19) United States
(12) Plant Patent Application Publication
(10) Pub. No.: US 2025/0386751 P1
(43) Pub. Date: Dec. 18, 2025

(54) Echinacea Plant Named 'BullEchipur 120'

(50) ;

(71) Applicant: Hartwig Bull, Gönnebek (DE)

(72) Inventor: Hartwig Bull, Gönnebek (DE)

(21) Appl. No.: 19/235,179

(22) Filed: Jun. 11, 2025

Related U.S. Application Data
(60) Provisional application No. 63/731,837, filed on Jun. 12, 2024.

(51) Int. Cl. A01H 6/14; A01H 5/02

(52) U.S. Cl. CPC A01H 6/1448; A01H 5/02
USPC PLT/428

(57) ABSTRACT

A new and distinct variety of Echinacea plant named ‘BullEchipur 120’ having a new color, large flower, good growth habit, more uniformity, good branching plant, and good propagation rates.

### CROSS REFERENCE TO RELATED APPLICATIONS

This application claims the benefit of priority from U.S. Provisional Patent Application No. 63/731,837, filed Jun. 12, 2024, which is incorporated herein by reference in its entirety.

Botanical classification: Echinacea purpurea.

Varietal denomination: ‘BULLECHIPUR 120’.

### BACKGROUND OF THE INVENTION

The present invention relates to a new and distinct cultivar of Echinacea, botanically known as Echinacea purpurea, and hereinafter referred to by the name ‘BullEchipur 120’.

The new Echinacea was discovered by the Inventor in August 2021, in a controlled environment in Gönnebek, Germany, as a part of a planned breeding program. The new Echinacea was selected on the basis of a new color, large flower, good growth habit, more uniformity, good branching plant, and good propagation rates.

Asexual propagation of the new cultivar by in vitro culture since September 2021, in Gönnebek, Germany, has shown that the unique features of this new Echinacea are stable and reproduced true to type in successive generations.

### SUMMARY OF THE INVENTION

The following traits have been repeatedly observed and are determined to be unique characteristics of ‘BullEchipur 120’. These characteristics in combination distinguish ‘BullEchipur 120’ as a new and distinct cultivar:
1. Compact growth
2. Watermelon flower color
3. Large flowers
4. More branching habit
5. Very uniform growth
6. Nice green leaves
7. Fast crop time
8. Early flowering

In side-by-side comparisons conducted by the Inventor in Gönnebek, Germany, plants of the new Echinacea differed from plants of the female parent, ‘Bul 2020-14’ of Echinacea purpurea, in the following characteristics:
1. More compact
2. Watermelon flower color
3. Larger flowers
4. Medium branching
5. Green clean leaves
6. Earlier flowering

In side-by-side comparisons conducted by the Inventor in Gönnebek, Germany, plants of the new Echinacea differed from plants of the male parent, ‘Bul 2020-21’ of Echinacea purpurea, in the following characteristics:
1. More compact
2. Watermelon flower color
3. More branches
4. Earlier flowering

Plants of the new Echinacea can be compared to the plants of the Echinacea cultivar ‘Cheyenne Spirit’, believed to be unpatented. In side-by-side comparisons conducted by the Inventor in Gönnebek, Germany, plants of the new Echinacea differed from plants of the cultivar ‘Cheyenne Spirit’ in the following characteristics:
1. More compact
2. Watermelon flower color
3. More branching habit
4. Green clean leaves
5. Even branching habit

### DESCRIPTION OF THE DRAWINGS

The accompanying colored photographs illustrate the overall appearance of the new Echinacea, showing the colors as true as it is reasonably possible to obtain in colored reproductions of this type. Colors in the photographs may differ slightly from the color values cited in the detailed botanical description, which accurately describes the colors of the new Echinacea.

FIG. 1 comprises a top perspective view of a typical plant of ‘BullEchipur 120’ shown in natural lighting.

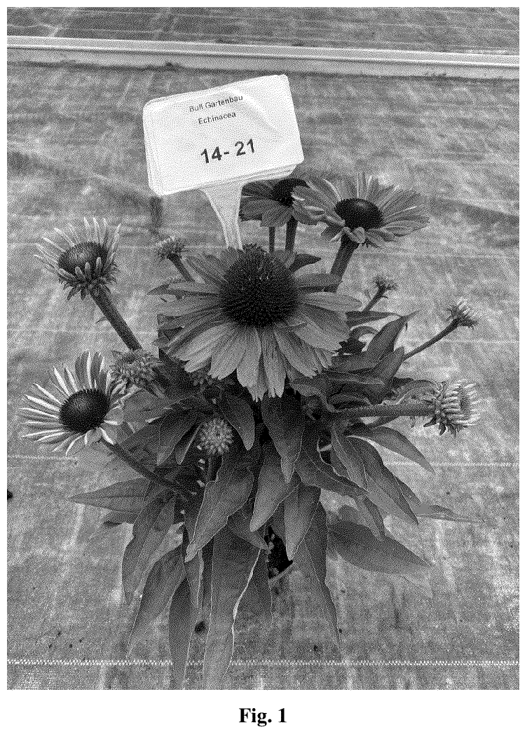

### DESCRIPTION OF THE PLANT

The cultivar Echinacea has not been observed under all possible environmental conditions. The phenotype may vary somewhat with variations in environment such as temperature and light intensity without, however, any variance in genotype.

The aforementioned photographs and following observations and measurements describe plants of the new Echinacea that were grown in an outdoor nursery in Gönnebek, Germany. During the production of the plants, temperatures averaged about 18-20° C. Plants used for the photographs and description were about 20 weeks from planting. Color references are made to The Royal Horticultural Society Colour Chart, 2015 version, except where general terms of ordinary dictionary significance are used.
Plant:
   Botanical classification.—Echinacea purpurea.
   Parentage.—‘Bul-2020-14’ and ‘Bul-2020-21’.
   Propagation.—Time to initiate roots: About 7 days. Time to develop roots: About 40-50 days. Time to produce a finished flowering plant: About 12 weeks. Root description: Lots of roots, free-branching, nice, healthy-looking root system.
   Plant description.—Growth rate/vigor: Compact, upright. Plant height: 35-40 cm. Plant width: 23-30 cm. Branching habit: Good branching, upright, shows a compact plant. Lateral branches: Number per plant: 12-20. Length: 2-25 cm. Diameter: 3-8 mm. Angle: Upright, outward. Strength: Strong. Texture: Pubescent. Color: 146C. Internode length: 1-5 cm. Foliage: Length: 3-20 cm. Width: 1-5.5 cm. Shape: Lanceolate. Shape of Apex: Acuminate. Shape of Base: Attenuate to Acute. Aspect: Straight to curved. Texture: Upper surface: Rough. Lower surface: Rough. Young leaves: Upper surface color: 137A. Lower surface color: 137C. Fully expanded leaves: Upper surface color: 137A. Lower surface color: 137C. Venation: Type: Cross-venulate to longitudinal. Color: Upper surface: 145A. Lower surface: 145B. Margin: Entire.
   Flowers.—Bud: Stage of opening: Single type blossom. Shape: Elliptic shaped ray florets and tubular disk floret. Diameter: 1-3.5 cm. Length: 1.2-2.5 cm. Color: When closed: N137C. When open: Natural Flowering Season: 10-12 weeks after planting. Inflorescence: Form: Single flower. Disc and ray floret arrangement: Single composite flower. Average number per plant: 10-20. Average number per branch: 1-3. Fragrance: None. Diameter: 4-11 cm. Height: 2-4 cm. Diameter of disc: 1.5-4 cm. Receptacle height: 1 cm. Receptacle diameter: 1.5-4 cm. Ray florets: Number per inflorescence: 15-23. Arrangement: In two or three whorls. Length: 1.5-5 cm. Width: 0.9-1.6 cm. Shape: Ellipse. Shape of Apex: Obtuse. Shape of Base: Acute. Margin: Entire. Texture: Upper surface: Smooth, glabrous. Lower surface: Rough. Color: When opening: Upper surface: 54B. Lower surface: 54C. At maturity: Upper surface: 54B. Lower surface: 54C. Ground color description: Watermelon. Disc florets: Number per inflorescence: Numerous, too many to count. Arrangement: In center of receptacle. Length: 8-15 mm. Width: 1-2 mm. Shape: Tubular. Shape of Apex: Sharp. Color: Immature: 185A. At maturity: 45A. Phyllaries: Number per inflorescence: 35-50. Arrangement: In 3-4 whorls. Length: 0.5-1.5 cm. Width: 2-3 mm. Shape: Lanceolate. Shape of Apex: Elongate, oval. Shape of Base: Fused. Margin: Entire. Texture: Upper surface: Rough. Lower surface: Rough. Color: Immature: Upper surface: NN137C. Lower surface: NN137C. At maturity: Upper surface: NN137C. Lower surface: NN137C.
   Reproductive organs.—Androecium: Presence: On disk florets. Number: One. Filament: Length: 1-2 mm. Color: 45A. Anther: Shape: Oval. Length: 1 mm. Color: 45A. Pollen: Color: 15B. Amount: Too much to quantify. Gynoecium: Presence: In disk and ray florets. Pistil length: 1-2 mm. Stigma: Shape: Two parted. Color: 45A. Style: Length: 1-2 mm. Color: 45A. Seeds/fruit: Length: 1-2 mm. Color:
   Disease/pest resistance.—Disease resistance: No additional disease resistance has been observed beyond the typical resistance in Echinacea plants. Pest resistance: No additional pest resistance has been observed beyond the typical resistance in Echinacea plants.
   Weather tolerance.—Temperature tolerance: No additional temperature resistance has been observed beyond the typical resistance in Echinacea plants. Drought tolerance: No additional drought resistance has been observed beyond the typical resistance in Echinacea plants.

I claim:

1. A new and distinct cultivar of Echinacea plant named ‘BullEchipur 120’, as illustrated and described.
